(19) United States
(12) Patent Application Publication
(10) Pub. No.: US 2024/0317018 A1
(43) Pub. Date: Sep. 26, 2024

(54) SYSTEM AND METHOD FOR MONITORING AND CONTROLLING A PROCESS FOR SANITISING PASSENGER COMPARTMENTS

(71) Applicant: IPAM TECHNOLOGY S.A., Santiago (CL)

(72) Inventor: Carlos Antonio LIMA ARAVENA, Santiago (CL)

(21) Appl. No.: 18/260,070

(22) PCT Filed: Dec. 30, 2020
(86) PCT No.: PCT/CL2020/050201

§ 371 Date: Dec. 14, 2023

(51) Int. Cl. B60H 1/00; A61L 9/22

(52) U.S. Cl. CPC B60H 1/00657; A61L 9/22; A61L 2209/111

(57) ABSTRACT

The invention refers to the monitoring and control of cabins sanitization process. In particular, the invention relates to a remote monitoring and control system and method to monitor and control a cabin sanitization process using a portable sanitation device.

The invention refers to the monitoring and control of a cabin sanitization process. In particular, the invention refers to a remote monitoring and control system and method, to monitor and control a cabin sanitization process, using at least one portable sanitization device.

The main objective of the invention is to implement a sanitization process to reliably purify and sanitize the interior space of one or more cabins, using at least one portable sanitization device that sanitizes said interior space through the ionization of the air that circulates through said device.

In order to guarantee reliable sanitization of the interior space of each cabin, the sanitization process requires an accurate control of an operating sequence of each portable sanitizing device, given by an operation cycle of each device. Therefore, the implementation of the sanitization process using the system and method of the invention seeks to ensure compliance with each operating sequence.

Specifically, the proposed solution solves problems associated with quality assurance (Quality Assurance, QA) and quality control (Quality Control, QC) of a sanitization process resulting from non-compliance with the operating sequence of one or several portable sanitization devices used in said sanitization process.

### BACKGROUND

In the industry, whether in the productive, manufacturing or service sectors, quality management is a common problem to solve. To do this, the usual solution means implementing rules, protocols and other tools aimed at identifying failures in products and/or processes to improve the level of performance and ensure reliable products and/or services.

In this context, the industry associated with sanitization requires quality management with the strictest standards, since consumers are more sensitive to any failure in the associated product and/or service, failures that can even mean serious health problems in the consumers. For this reason, providers of health-related products and/or services, for example, the sanitization of living spaces seek high quality standards, ensuring compliance with all necessary measures to deliver a reliable product and service.

Among the technological tools most used in quality management are monitoring and control systems, which implement technical and computational solutions specially designed and adapted to each process to be monitored and controlled. By way of example, document U.S. Pat. No. 9,564,038B2 describes a method and apparatus for improving hand sanitization compliance in healthcare facilities, using a sanitization station that communicates wirelessly with a monitoring device carried by the user adapted to count time elapsed since the last hand washing. The solution in U.S. Pat. No. 9,564,038B2 is a clear example of the implementation of information technologies in a problem associated with sanitization with a design specially configured for the reality of the problem of handwashing of health personnel, ensuring acceptable levels of reliability in said technical field. Another example is exhibited in document EP2766833B1 which describes a method and system for monitoring/compliance with sanitization protocols using identifying labels, for example, through a radio-frequency identification technology (Radio-Frequency Identification, RFID), either in a patient and/or in a user or staff with the task of sanitizing a space or utensils and implementing a sanitization verification system capable of reading said labels and verifying compliance with a sanitization protocol.

Although the previous documents are directly associated with the health sector, there are other technical solutions that use sanitization processes in different areas, such as food. In this context, document U.S. Pat. No. 7,976,777B2 discloses a packaging set with autonomous monitoring aimed at monitoring the presence of a sanitizing agent such as ozone inside the packaging and in contact with an object or product to be sanitized, such as food. This solution uses sensors, processors and communication media aimed at carrying out monitoring communicating it to a user and/or storing the associated data in a database, either in systems implemented in the packaging set or in a network with remote access.

In view of the above, it is evident that providing systems and methods for monitoring and controlling sanitization processes is a necessity with various solutions specially designed according to each sanitization application. For this reason, in the technical field of sanitizing living spaces, that is, spaces intended to be inhabited permanently or temporarily, such as the interior of a vehicle or cabins of a home or any place of accommodation, it is necessary to have technical solutions that ensure the reliability of the sanitization process, implementing methods and systems specially designed for the particularities associated with the sanitization of cabins.

As an example, there is a need to monitor and control sanitization processes of vehicle interiors, as would be the case of sanitizing rental vehicles which are used by different people or after private vehicles maintenance and/or repair operations have been carried out which require access to the interior of the vehicles by garage personnel. In both cases, the sanitization of the cabins is of great help and provides added value to the corresponding services in these examples, ensuring that customers access their own or leased vehicles with a sanitized interior space. In this regard, currently exist different technologies and are used to sanitize living spaces, such as vehicle cabins, but there are no monitoring and control systems or methods specially designed to ensure the correct operation of said technologies.

In this context, cabin sanitization has a series of particularities that make the sanitization process very different from others. Among others, these particularities can be associated with the amount of sanitization equipment or devices available, the number of cabins to be sanitized, the volume of each cabin, the minimum sanitization time for each cabin, daily goals for processed cabins, etc. As an example, the system and method of the invention may be implemented in a mechanical workshop where sanitization devices are used prior to delivering the vehicles to customers sanitizing the interior space of each vehicle. In this example, it is a common practice to prematurely remove the sanitization device from one cabin to enter it in another before the device's operating sequence is completed seeking to accelerate the delivery of vehicles at the expense of the sanitization process. This results in low quality or even insufficient sanitization, since the minimum time required for correct sanitization of the interior space of each cabin is not ensured. Therefore, the system and method of the invention seek to ensure the correct application of the sanitization process in each cabin or at least record and/or alert the performance of partial or incomplete sanitization processes which do not ensure the quality of sanitization.

On the other hand, for an end user who receives the sanitization service it is not easy to verify if the sanitization process was carried out correctly, since it would require testing the presence of pathogens inside the sanitized cabin. Furthermore, the sanitization services are usually carried out by intermediaries or user operators who are only responsible for executing the sanitization process but do not correspond to the end users of the sanitized cabins or the owner of the sanitization technology used. Due to the above, the reception of cabins without sanitization or with an incomplete sanitization process is common, without the end user being able to realize it. Therefore, for the owners of the sanitization technology it is a necessity to ensure a correct implementation of the sanitization process by any user, at least registering and/or alerting the incorrect use of the technology seeking that the end user access to correctly sanitized cabins with the associated technology.

The combination of the particularities specific to the technical field of the invention mentioned above, require a solution specially designed for the monitoring and control of the cabin sanitization process which avoids the problems indicated above and ensures the quality of the sanitization process.

Therefore, it is necessary to have a remote monitoring and control system and method that provides a comprehensive solution to the main problems involved in the cabin sanitization process, in accordance with the above mentioned.

### DESCRIPTION OF THE INVENTION

The proposed solution consists of a remote monitoring and control system for a cabin sanitization process. For this purpose, the system includes:
a plurality of cabins, wherein each cabin includes an interior space to be sanitized;
at least one portable sanitization device comprising at least one reaction chamber configured to be arranged in the interior space of at least one cabin and to purify and sanitize said interior space by ionizing air in said at least one reaction chamber;
at least one power source arranged to power the at least one portable sanitization device;
at least one communication unit arranged in data communication with the at least one portable sanitization device;
at least one control unit arranged to monitor and control an operating sequence of the at least one portable sanitization device; and
at least one server remotely arranged in a wireless communications network and in data communication with the at least one communication unit.

Pursuant to the foregoing, the operating sequence of the at least one portable sanitization device comprises:
a power-on action of the at least one portable sanitization device;
an operating time of the at least one portable sanitization device; and
a power-off action of the at least one portable sanitization device.

Furthermore, the at least one control unit is in data communication with at least one location unit integrated in the at least one portable sanitization device to obtain location data of the portable sanitization device. On the other hand, the operation sequence, and the location data, which are called the set of monitored operation data, are communicated to the at least one server through the at least one communication unit. Finally, the at least one server is configured to compare the set of monitored operation data with a set of predefined operation data, and to execute control actions over the operation of the at least one portable sanitization device based on said comparison and through the at least one control unit.

The proposed solution also consists of a method of monitoring and controlling a cabin sanitization process remotely. For this purpose, the method includes:
a) arranging at least one portable sanitizing device in a cabin of a plurality of cabins, wherein each cabin comprises an interior space to be sanitized and where the at least one portable sanitizing device comprises at least one reaction chamber to purify and sanitize said interior space by ionizing air in said at least one reaction chamber;
b) energizing the at least one portable sanitization device using at least one energy source;
c) starting an operating sequence of the at least one portable sanitization device, which comprises:
   a power-on action of the at least one portable sanitization device;
an operating time of the at least one portable sanitization device; and
   a power-off action of the at least one portable sanitization device;
d) monitoring and controlling the sequence of operation of the at least one portable sanitization device through at least one control unit;
e) obtaining location data from the portable sanitization device by means of the at least one control unit in data communication with at least one location unit integrated into the at least one portable sanitization device;
f) communicating the operating sequence and location date, called set of monitored operating data to at least one server through at least one communication unit arranged in data communication with the at least one portable sanitization device, wherein the at least one server is remotely arranged on a wireless communications network and in data communication with the at least one communication unit;
g) comparing the set of monitored operation data with a set of predefined operation data using the at least one server; and
h) executing control actions on the operation of the at least one portable sanitization device based on said comparison and through the at least one control unit.

According to one embodiment of the invention, the at least one communication unit and the at least one control unit can be integrated into the at least one portable sanitization device, either independently or forming a single communication and control unit. That is, each portable sanitization device can have its own communication and control unit.

According to one embodiment of the invention, the system and method may further comprise the assignment, by the at least one server or the at least one control unit, of a unique identifier to the set of monitored operation data of the at least one portable sanitization device. Furthermore, the system and method may comprise the registration through the at least one server of said set of monitored operation data and the assigned unique identifier in at least one database. To this end, the set of monitored operation data can be stored, in real time, during the operating sequence in a storage unit or internal memory of each portable sanitization device and upon completion of the sanitization process, said set of stored monitored operation data may be communicated to the at least one server for its association with the unique identifier and subsequent storage in at least one database. Said at least one database can be found remotely in association with the at least one server, either stored on said server on a remote access point and/or in the cloud. Alternatively, the monitored operating data may also comprise date and time of each on and off action of each operating sequence. On the other hand, the storage unit or internal memory of each portable sanitization device may be a non-volatile reading memory, for example, of the EEPROM type which may be erased electronically, at least in part, after obtaining the data stored in it.

According to one embodiment of the invention, the system and method may further comprise complementing the set of monitored operating data through the at least one server or the at least one control unit with a unique identifier of each cabin that has been subjected or will undergo the sanitization process. Preferably, said unique identifier of the cabin is obtained automatically, for example, by reading a code or label identifying the cabin previously assigned, such as an RFID label, or by communicating data with the cabin, for example, through the connection of at least one portable sanitization device with a communication port arranged in relation to the cabin. If the cabins are vehicle cabins said communication port may correspond to an OBD (On-Board Diagnostics) type port, which communicates the VIN (Vehicle Identification Number) of each vehicle to the portable sanitization device for its incorporation into the monitored operation data. In accordance with this embodiment, the operator user of each portable sanitization device will be responsible for connecting said device with the communication port of the cabin. Furthermore, in this scenario and optionally, the portable sanitization device may be configured to block the start of the sanitization process if it is not detected a data communication with said communication port ensuring the correct communication between the cabin and the device.

According to one embodiment of the invention, the system and method may also comprise the coordination, by means of the at least one server, of the operation of at least two portable sanitization devices in a synchronized manner, either in the same cabin or in two cabins different from the plurality of cabins. Said synchronization may correspond to a simultaneous or out of phase operation of the portable sanitization devices and may depend on the manipulation of said devices by an operator and/or the characteristics of the interior space (s) to be sanitized.

According to one embodiment of the invention, and once the set of monitored operation data is communicated to the at least one server for analysis and/or processing, the system and method may further comprise the issuance of an alarm, by means of the at least one server when at least one monitored operation data of the set of monitored operation data differs from at least one respective predefined operation data of the set of predefined operation data. That is, said alarm may be issued when an inconsistency is detected between monitored operation data and predefined operation data, for example, with respect to an ideal sanitization process. Alternatively, said alarm may be communicated remotely to the portable sanitization device for the issuance of a visual and/or audible alert in real time once the inconsistency between the data is detected, or it may be communicated in the form of a message to users' equipment, such as computers or telephones, through the communications network. By means of this alarm the operator users may execute corrective actions for the correct execution of the sanitization process, and supervisor users, such as the owners of the sanitization technology, may confirm the correct operation of the portable sanitization device (s).

As an example, the issuance of an alarm could occur in relation to the location of each portable sanitization device. In this example, the technology can issue an alarm if the location of a portable sanitization device, as part of the set of monitored operating data, does not correspond to a predefined location for said portable sanitization device. In this case, said predefined location may be determined based on the access of an operator user, who enters the predefined location of each portable sanitization device depending on the sequence of operation of the device and the daily sanitization plan, or through a pre-established location for each sanitization device by the technology owner. Such predefined location must consider the limitations of the localization unit, such as the accuracy of the localization mechanism.

Alternatively, the alarm may be triggered based on the sequence of operation and/or depending on its merit, upon detecting a movement action of the portable sanitization device. For example, the alarm may be triggered if the operating sequence and/or operating cycle has ended unexpectedly before completing a cycle according to the predefined operating data and/or if, by means of motion sensors such as optional accelerometers incorporated into a portable sanitization device, it is detected that said sanitization device has been moved by a user before completing the operation cycle. According to another alternative, an alarm may be triggered to indicate that the operating cycle has ended, in order to remove the portable sanitization device from the cabin. This alternative may be complemented with the embodiment that establishes a data connection between the cabin and the portable sanitization device, by means of a communication port, for example, issuing alarms indicating repeated applications of the sanitization process in the same cabin in a certain period of time.

According to another embodiment, the issuance of an alarm may be configured directly based on the monitored operation data once they are communicated to the at least one server. For example, the emission of said alarm may be configured according to a number of completed operation cycles, date and/or start/stop time of a portable sanitization device, among others. In this sense, the issuance of alarms may be linked to the control actions executed by the system and method of the invention once the monitored operation data has been analyzed or processed as a warning or indication to users regarding the execution of such control actions.

In this way, the association between the location of the portable sanitization device (localization data) with its operating sequence and/or with the cabin to be sanitized forming the set of monitored operation data, allows processing said information not only seeking to determine whether the operating sequence was fulfilled or not but also allowing to determine whether the sanitization device has moved or not in relation to the state of the operating sequence and the sanitized cabins. Then, in addition to seeking ways to ensure the correct use of the portable sanitization device, the invention is also capable of providing information that allows optimizing the use of said device in the field. For example, operator users of portable sanitization devices according to the invention can use the monitoring and control capabilities of the invention together with the analysis and processing of the monitored operation data to improve internal processes associated with the operation of the technology, for example, seeking to avoid or at least reduce downtime between sanitization processes. In this case, the invention can deliver information that allows optimizing not only the use of said sanitization devices, but also the entire process in which said device is involved.

According to one embodiment of the invention, the sequence of operation of the at least one portable sanitization device may be determined based on the volume or size of the interior space to be sanitized, wherein:
the volume of the interior space can be predefined, for example, by defining one or more average sizes or volumes of cabins to be sanitized, for example, associated with vehicle sizes; and/or
the volume of the interior space may be measured by one or more volumetric sensors arranged in the interior space, for example, integrated into the portable sanitization device and/or arranged in each cabin.

According to one embodiment of the invention, the system and method may further comprise the deployment, by the at least one control unit, of some or all of the monitored operation data of the portable sanitization device on a display unit, such as a LCD or LED screen, including at t least one portable sanitization device. Additionally, part or all the monitored operation data of the portable sanitization device may be communicated to the at least one server, for deployment and displaying on user equipment, such as computers or telephones, through the access to the communications network.

According to one embodiment of the invention, the monitored operation data may further comprise a number of operation cycles of the portable sanitization device, wherein each operation cycle comprises the operation sequence. The operating cycle may also include, together with the operating sequence, a rest time wherein the portable sanitizing device has stopped operating but the cabin is closed, that is, wherein the portable sanitizing device has not been removed from the cabin. Furthermore, according to the present embodiment, the invention may consider that:
each cabin corresponds to the cabin of a vehicle as the preferred cabin for the application of the invention;
the at least one power source corresponds to a power source integrated into each vehicle, for example, through a 12V electrical outlet that vehicles usually have or a 24V electrical outlet that larger vehicles such as trucks or buses usually have; and
each operation cycle includes an operating time of between 15 to 20 minutes and a rest time of between 1 to 3 minutes, preferably, the operation time is 18 minutes, and the rest time is 2 minutes for cabins of one average size of 2 m³.

In relation to the above, the system and method of the invention may comprise the following operations:
connecting, within each cabin, the at least one portable sanitization device to the vehicle's power source;
starting an operation cycle, circulating air from inside the cabin through the at least one reaction chamber and out of the portable sanitization device by means of at least one unit for moving air included in the at least one portable sanitization device and recirculating air inside the cabin through the at least one reaction chamber; and ionizing the air that circulates through the at least one reaction chamber expelling ionized air into the interior space of the vehicle's cabin.

On the other hand, according to a preferred embodiment of the invention, the system and method of the invention may also operate together with a ventilation, air conditioning and/or air recirculation system of the cabin itself, a system that must be activated to operate in conjunction with the portable sanitization device. For example, in the vehicle cabins sanitization embodiment, the system and method of the invention may comprise the activation, preferably at maximum power and minimum temperature before or during each operating cycle, of an air recirculation system comprised in each vehicle. Said air recirculation system may correspond to the ventilation system of each vehicle adjusted for air recirculation and, preferably, at maximum power and minimum temperature, with activated air conditioning if available. In this context, the activation of the recirculation system is carried out manually by an operator who manually activates the vehicle's ventilation or air conditioning system adjusting it to the minimum temperature at its maximum power and in recirculation mode. Alternatively, and if the communication with the vehicle allows it, the activation of the recirculation system with the desired settings can be carried out remotely by means of a signal sent to a controller of each vehicle, for example, through the communication port of the vehicle. The activation of the ventilation system of each cabin, in air recirculation mode, seeks to generate an air flow that circulates inside the cabin, said air flow with characteristics that favor the sanitization process seeking to ensure that said flow contact the existing surfaces both inside the ventilation system and inside the cabin optimizing its sanitization.

Finally, according to one embodiment of the system and method of the invention, the control actions executed on the operation of the at least one portable sanitization device based on the comparison between the monitored and predefined operation data sets, comprise:
blocking/unblocking a portable sanitization device aimed at preventing/allowing a new cycle of said device operation;
assigning a predefined number of the sanitization process applications or operation cycles to a portable sanitization device aimed at allowing said device to operate for a certain number of times; or
releasing a portable sanitization device aimed at allowing said device to function in a released manner.

The above control actions, which allow controlling the operation of portable sanitization devices through management of the operation of said devices, in addition to being used in the operational environment of the sanitization process, may be used as tools that rule a commercial relationship between the owner of the technology and its users. For example, such control actions can be linked to limits or goals of the technology use previously agreed between the owner and user operators of the technology.

Alternatively, the invention contemplates the potential to implement other control actions associated with the operation sequence and operation of each portable sanitization device, for example, remote start/shutdown of a portable sanitization device; continuous monitoring during each operation cycle through appropriate monitoring and communications units; adjustment of operating parameters in real time, such as delayed start/shutdown of a portable sanitization device, duration of operating time, duration of standby time, adjustment of power and operating mode of the cabin ventilation system and/or to measure cabin volume using associated sensors; stop/activate recording of monitored operation data on the internal and/or external storage unit; stop/activate sending the set of monitored operation data to a server; lock/unlock a portable sanitization device based on contractual agreements, etc. Ultimately, the capabilities of the system and method of the invention may vary depending on the devices and components that are integrated into it obtaining a solution that may comprise a simple recording of monitored operation data up to an advanced monitoring and control completely remote and practically in real time.

### BRIEF DESCRIPTION OF THE FIGURES

As part of the present invention, the following representative figures are exhibited which show preferred embodiments of the invention and, therefore, should not be considered as limitations to the definition of the claimed subject matter.

FIG. 1 shows a diagram of the system of the invention according to an exemplary embodiment.

FIG. 2 shows a diagram of the system of the invention according to a preferred embodiment.

### DETAILED DESCRIPTION OF THE INVENTION EMBODIMENTS

In relation to what was previously described in the Description of the Invention section, in this section more specific aspects of some embodiments of the invention are described, exhibited as non-limiting examples thereof. To do this, reference is made to the main aspects of the proposed solution.

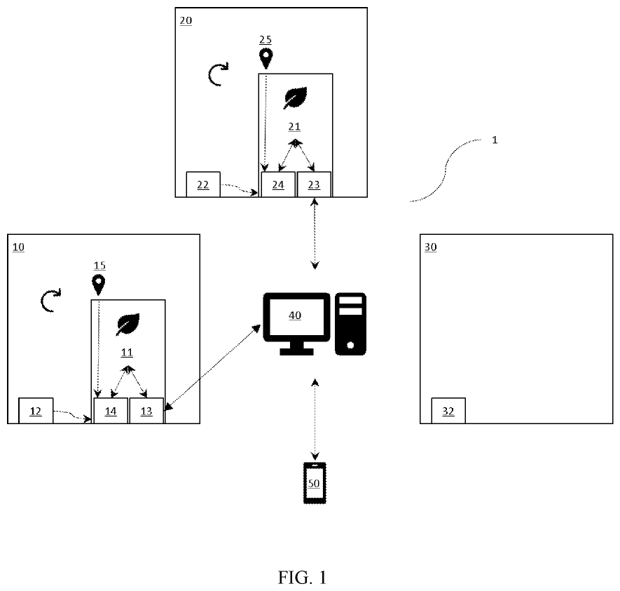

FIG. 1 shows a diagram of the monitoring and control system (1) according to one embodiment of the invention, being possible to appreciate that said system is formed, in its general aspects, by three cabins (10, 20, 30) with an interior space to be sanitized, a server (40) arranged remotely in a wireless communication network and a user equipment (50) arranged in communication with the server (40) by means of said wireless communication network. As seen in FIG. 1, the monitoring and control system (1) also consists of two portable sanitization devices (11, 21) which in this case are shown arranged inside the cabins (10, 20) in order to sanitize the interior space of the cabins (10, 20), respectively.

Furthermore, in FIG. 1 it is possible to appreciate that each sanitization device (11, 21) is connected to an energy source (12, 22) which, in the embodiment shown, is independent of each device. Alternatively, each device may comprise its own power source or be connected to a common power source, if said power source meets the power requirements of each device. In FIG. 1 it is also appreciated that said devices are shown in data communication with a communication unit (13, 23) and with a control unit (14, 24) which may be integrated into each sanitization device (10, 20) making them autonomous during their operation. In fact, embodiments of the invention allow the completely autonomous operation of the portable sanitization devices only with the recording of the monitored operation data in an internal and/or external storage unit for communication to the server (40) subsequently, once an operating cycle has been completed. It is also possible to appreciate that the portable sanitization devices (11, 21) comprise a location unit (15, 25) arranged in data communication with the control unit (14, 24). In this way, each control unit (14, 24) receives location data of each device from the location units (15, 25), data that is combined with the operating sequence that is monitored by each control unit (14, 24) forming a set of monitored operation data that is communicated through each communication unit (13, 23) to the server (40). According to the preferred embodiment, the communication between each portable sanitization device (11, 21) and the server (40) may occur after the end of the operation cycle or operation sequence, that is, at the end of each application or sanitization process. In this context, a user of the monitoring and control system (10) who has authorization to do so, may access the set of operational data monitored remotely and in real time, either through direct access to the server (40) or through access from the user equipment (50).

Finally, in FIG. 1 and in relation to the cabin (30) which does not include a sanitization device and which is waiting for sanitization by any of the portable sanitization devices (11, 21) or which has already completed the sanitization process, it is possible to appreciate that the energy source (32), according to one embodiment, is external to the device and may be part of the same cabin or may be integrated therein. In said embodiment, a portable sanitization device is connected to the energy source (32) once it has been positioned inside the cabin (30) before starting the operating sequence.

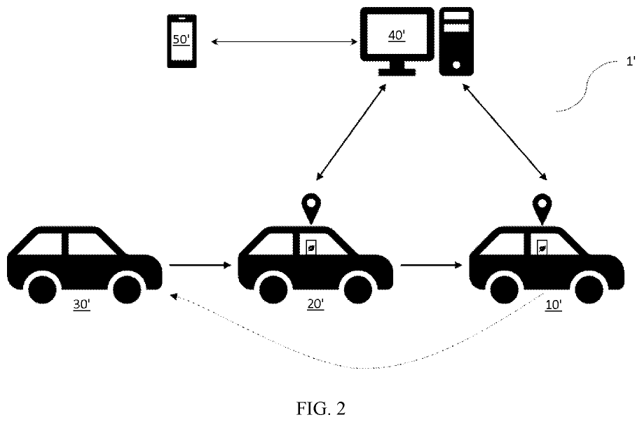

On the other hand, FIG. 2 shows an embodiment of the monitoring and control system (1′) specially exhibited with respect to the sanitization of vehicle cabins. In FIG. 2 a series of vehicles can be seen in a processing line, wherein a first vehicle (10′) and a second vehicle (20′) have a portable sanitization device in their cabin. Said first and second vehicles (10′, 20′) are in a sanitization process that is monitored and controlled remotely through the server (40′) which receives sets of monitored operation data from each portable sanitization device, preferably once each operating cycle has finished. In this sense, once the server (40′) has detected that at least one of the portable sanitization devices has finished its operating cycle it may issue an alarm on a user equipment (50′), such as a message text and/or in the portable sanitization device itself as a light signal, so that an operator user removes the portable sanitization device that has finished its cycle, in this example the one that is arranged in the first vehicle (10′), to place it inside the next cabin to be sanitized, in this example, inside a third vehicle (30′), thus optimizing the sanitization operation and the processing line. Alternatively, the end of the operation cycle is signaled by each portable sanitization device autonomously, without requiring communication with the server, by means of the at least one control unit arranged in relation to each portable sanitization device.

In relation to the above, some scopes of the main components of the present invention are exhibited below as a complement to the definitions previously presented.

### Cabins

As noted above, in the context of the invention, the cabins correspond to any space intended to be inhabited permanently or temporarily. According to preferred embodiments of the invention, the cabins correspond to spaces that are closed or that can be closed generating an interior space where the environment can be controlled, for example, the cabin of a vehicle or a cabin in a house or hotel. Said interior space, being closed or capable of being closed, is susceptible to a sanitization process using at least one portable sanitization device through the ionization of the air that circulates through said device.

Preferably, the invention relates to the sanitization of vehicle cabins which comprise an energy source with access through a 12V or 24V electrical outlet depending on the type of vehicle. Said energy source is sufficient to power a portable sanitization device according to the invention.

Furthermore, vehicle cabins are characterized by generating a closed interior space that, depending on the vehicle model, has an average volume of 2 m³. Then, the at least one portable sanitization device is designed to sanitize, in a defined operating time, said volume. Notwithstanding the foregoing and according to alternative embodiments, the size of the cabins may be larger, and portable sanitization devices specially designed for said cabins must be used, two or more devices per cabin or according to one embodiment, it may be necessary to adjust the operating time of the devices depending on the volume to be sanitized either at the level of manual configuration of each sanitizing device or through volumetric sensors to measure the size of the cabin and, based on the same, adapt the operating time of one or more portable sanitization devices.

### Portable Sanitization Device

In this description, some of the essential details of the portable sanitization device have already been provided, being relevant to highlight that preferably, it is a compact device that is available to carry out a sanitization process inside a cabin through ionization of the air circulating through the device. For this purpose, the portable sanitization device includes at least one reaction chamber inside where the ionization process of the circulating air begins. Furthermore, according to embodiments of the invention, the portable sanitization device comprises at least one unit for moving air arranged to force air into a cabin through the at least one reaction chamber.

On the other hand, the portable sanitization device comprises at least one location unit integrated into said device, such as a GPS or GLONASS antenna, in order to obtain location data from said device. Furthermore, the portable sanitization device is in communication with at least one communication unit, at least one control unit and at least one server for monitoring, processing, and communicating operation data of said device. According to a preferred embodiment, both the at least one communication unit and the at least one control unit are located inside the portable sanitization device ensuring that said device can operate autonomously.

Alternatively, the portable sanitization device may also comprise a display unit, for the deployment of part or all the operating data of said device, and/or a storage unit for recording part or all the operating data.

### Communication Unit

The communication unit is a component of the invention intended to receive and transmit data between at least one portable sanitization device and at least one server, either during the sanitization process or once the operation cycle has finished. In particular, the communication unit is responsible for sending monitored operation data to the at least one server and is configured to receive control instructions or actions from said at least one server, for the execution of said actions by the portable sanitization device.

Preferably, the communication unit is wireless and is integrated into the portable sanitization device.

### Control Unit

The control unit is a component of the invention intended to monitor and control the operation data of the portable sanitization device, in order to record said operation data and execute control actions received from at least one server.

Preferably, the control unit is integrated into the portable sanitization device and corresponds to an electronic circuit with a programmed microprocessor, in order to manage the operating sequence of said device and execute the control actions. The control unit is arranged to control the operation of all the electrical and electronic components of the portable sanitization device, either through preconfigured instructions or through control actions received through the communication unit.

### Server

The server, which is remotely arranged on a wireless communications network and in data communication with at least one portable sanitization device, through the at least one communication unit, represents the computational analysis of the monitored operation data and received from said device. Preferably, said computational analysis comprises the comparison of sets of monitored operation data with sets of predefined operation data for the detection of inconsistencies between said data sets and the implementation of control actions.

In this context, the definition of the server includes computational processing, for example, in the cloud or directly on one or more computers for the processing of operation data and its treatment in one or more databases. In this way, the server allows the visualization of said operation data and, alternatively, the processing thereof through different known tools, for example, through a platform with web access or through direct communication with user equipment for the management of the operation data. The same structure is considered for the issuance and communication of alarms, both to the portable sanitization device and to the user equipment.

Preferably, the operation of the at least one server is closely related to the operation of the at least one control unit, the latter being the one that finally executes, on the portable sanitization device, the control actions issued by the at least one server.

1. A remote monitoring and control system for a cabin sanitization process, the system CHARACTERIZED in that it comprises:
a plurality of cabins, where each cabin includes an interior space to be sanitized;
at least one portable sanitization device comprising at least one reaction chamber configured to be arranged in the interior space of at least one cabin and to purify and sanitize said interior space by ionizing air in said at least one reaction chamber;
at least one power source arranged to power the at least one portable sanitization device;
at least one communication unit arranged in data communication with the at least one portable sanitization device;
at least one control unit arranged to monitor and control an operating sequence of the at least one portable sanitization device; and
at least one server remotely arranged on a wireless communications network and in data communication with the at least one communication unit;
wherein the operating sequence of the at least one portable sanitization device comprises:
a power-on action of the at least one portable sanitization device;
an operating time of the at least one portable sanitization device; and
a shutdown action of the at least one portable sanitization device;
wherein the at least one control unit is in data communication with at least one localization unit integrated in the at least one portable sanitization device, in order to obtain localization data of the portable sanitization device;
wherein the operation sequence and the localization data, which are called a set of monitored operation data, are communicated to the at least one server by means of the at least one communication unit; and
wherein the at least one server is configured to compare the set of monitored operation data with a set of predefined operation data, and to execute control actions over the operation of the at least one portable sanitization device based on said comparison and by means of the at least one control unit.

2. The system according to claim 1, CHARACTERIZED in that the at least one communication unit and the at least one control unit are integrated with the at least one portable sanitization device either independently or in a single communication and control unit.

3. The system according to any of claims 1-2, CHARACTERIZED in that the at least one server or the at least one control unit are configured to assign a unique identifier to the set of monitored operation data of the at least one portable sanitization device and in that the at least one server is configured to register said set of monitored operation data and the assigned unique identifier in at least one database.

4. The system according to any of claims 1-3, CHARACTERIZED in that the at least one server or the at least one control unit are configured to complement the set of monitored operation data with a unique identifier of each cabin.

5. The system according to any of claims 1-4, CHARACTERIZED in that it comprises at least two portable sanitization devices, wherein the at least one server is configured to coordinate the operation of said at least two portable sanitization devices in a synchronized manner, either in the same cabin or two different cabins of the plurality of cabins.

6. The system according to any of claims 1-5, CHARACTERIZED in that the at least one server is configured to issue an alarm when at least one monitored operation data of the set of monitored operation data differs from at least one respective predefined operation data from the set of predefined operation data.

7. The system according to any of claims 1-6, CHARACTERIZED in that the operating sequence of the at least one portable sanitization device is determined based on the volume of the interior space to be sanitized, wherein:
the volume of the interior space is predefined; or
the volume of the interior space is measured by one or more volumetric sensors arranged in the interior space.

8. The system according to any of claims 1-7, CHARACTERIZED in that the at least one portable sanitization device comprises a display unit, wherein the at least one control unit is configured to deploy part or all the monitored operation data of the portable sanitization device in said display unit.

9. The system according to any of claims 1-8, CHARACTERIZED in that the monitored operation data further comprises a number of operation cycles of the portable sanitization device, wherein each operation cycle comprises the operation sequence.

10. The system according to claim 9, CHARACTERIZED in that:
each cabin corresponds to the cabin of a vehicle;
the at least one energy source corresponds to an energy source integrated in each vehicle; and
each operation cycle includes an operating time of between 15 to 20 minutes and a rest time of between 1 to 3 minutes, preferably, the operation time is 18 minutes, and the rest time is 2 minutes for cabins of an average size of 2 m³;
wherein the at least one portable sanitization device comprises at least one unit for moving air, wherein the at least one unit for moving air is configured to circulate air from inside the cabin through the at least one reaction chamber and outside the portable sanitization device;
wherein, within each cabin the at least one portable sanitization device is configured to connect to the vehicle's energy source and initiate an operation cycle, recirculating air into the cabin through the at least one reaction chamber; and
wherein the at least one reaction chamber is configured to ionize the air that circulates through it, expelling ionized air into the interior space of the vehicle's cabin.

11. The system according to claim 10, CHARACTERIZED in that each vehicle comprises an air recirculation system configured to be activated, preferably at maximum power and minimum temperature, before or during each operation cycle, where the activation is manual by means of an operator user or remotely through a signal sent to a controller of each vehicle.

12. The system according to any of claims 1-11, CHARACTERIZED in that the control actions comprise:
locking/unlocking at least one portable sanitization device, preventing/allowing the start of a new operation cycle;
assigning a predefined number of sanitizing process applications or operation cycles to the at least one portable sanitizing device, allowing said at least one portable sanitizing device to operate for a certain number of times; or
releasing at least one portable sanitization device, allowing said at least one portable sanitization device to operate in a released manner.

13. A method of monitoring and controlling a cabin sanitization process remotely, the method CHARACTERIZED in that it comprises:
i) arranging at least one portable sanitization device in a cabin of a plurality of cabins, wherein each cabin comprises an interior space to be sanitized, and where the at least one portable sanitization device comprises at least one reaction chamber to purify and sanitize said interior space by ionizing air in said at least one reaction chamber;
j) energizing the at least one portable sanitization device, using at least one energy source;
k) starting an operating sequence of the at least one portable sanitization device, which comprises:
a power-on action of the at least one portable sanitization device;
an operating time of the at least one portable sanitization device; and
a shutdown action of the at least one portable sanitization device:
l) monitoring and controlling the operation sequence of the at least one portable sanitization device through at least one control unit;
m) obtaining location data from the portable sanitization device by means of the at least one control unit in data communication with at least one location unit integrated into the at least one portable sanitization device;
n) communicating the operating sequence and location data, which are called a set of monitored operating data, to at least one server through at least one communication unit arranged in data communication with the at least one portable sanitization device, wherein the at least one server is remotely arranged on a wireless communications network and in data communication with the at least one communication unit;
o) comparing the set of monitored operation data with a set of predefined operation data using the at least one server; and
p) executing control actions on the operation of the at least one portable sanitization device based on said comparison and through the at least one control unit.

14. The method according to claim 13, CHARACTERIZED in that the at least one communication unit and the at least one control unit are integrated with the at least one portable sanitization device, either independently or in a single communication and control unit.

15. The method according to any of claims 13-14, CHARACTERIZED in that it further comprises assigning a unique identifier to the set of monitored operation data of the at least one portable sanitization device through the at least one server or the at least one storage unit and in that it also comprises registering said set of monitored operation data and the assigned unique identifier in at least one database through the at least one server.

16. The method according to any of claims 13-15, CHARACTERIZED in that it also comprises complementing the set of monitored operation data with a unique identifier of each cabin by means of the at least one server or the at least one control unit.

17. The method according to any of claims 13-16, CHARACTERIZED in that it also comprises coordinating the operation of at least two portable sanitization devices in a synchronized manner, either in the same cabin or in two different cabins of the plurality of cabins by means of the at least one server.

18. The method according to any of claims 13-17, CHARACTERIZED in that it further comprises issuing an alarm when at least one monitored operation data of the set of monitored operation data differs from at least one respective predefined operation data of the set of predefined operation data through the at least one server.

19. The method according to any of claims 13-18, CHARACTERIZED in that the sequence of operation of the at least one portable sanitization device is determined based on the volume of the interior space to be sanitized, wherein:
the volume of the interior space is predefined; or
the volume of the interior space is measured by one or more volumetric sensors arranged in the interior space.

20. The method according to any of claims 13-19, CHARACTERIZED in that it further comprises deploying part or all the monitored operation data of the portable sanitization device in a display unit comprised in the at least one portable sanitization device by means of the at least one control unit.

21. The method according to any of claims 13-20, CHARACTERIZED in that the monitored operation data further comprises a number of operation cycles of the portable sanitization device, wherein each operation cycle comprises the operation sequence.

22. The method according to claim 21, CHARACTERIZED in that:
each cabin corresponds to the cabin of a vehicle;
the at least one energy source corresponds to an energy source integrated in each vehicle; and
each operation cycle includes an operating time of between 15 to 20 minutes and a rest time of between 1 to 3 minutes, preferably, the operation time is 18 minutes and the rest time is 2 minutes for cabins of an average size of 2 m³;
wherein the method also comprises:
connecting, within each cabin the at least one portable sanitization device to the vehicle's power source;
starting an operation cycle, circulating air from inside the cabin through the at least one reaction chamber and outside the portable sanitization device by means of at least one unit for moving air included in the at least one portable sanitization device portable, and recirculating air inside the cabin through the at least one reaction chamber; and
ionizing the air that circulates through the at least one reaction chamber expelling ionized air into the interior space of the vehicle's cabin.

23. The method according to claim 22, CHARACTERIZED in that it further comprises activating, preferably at maximum power and minimum temperature before or during each operating cycle, an air recirculation system included in each vehicle, wherein the activation is manual by an operator user, or remote through a signal sent to a controller of each vehicle.

24. The method according to any of claims 13-23, CHARACTERIZED in that the control actions comprise:
locking/unlocking at least one portable sanitization device preventing/allowing the start of a new operation cycle;
assigning a predefined number of sanitizing process applications or operation cycles to the at least one portable sanitizing device, allowing said at least one portable sanitizing device to operate for a certain number of times; or
releasing at least one portable sanitization device allowing said at least one portable sanitization device to operate in a released manner.
